(19) United States
(12) Patent Application Publication
(10) Pub. No.: US 2020/0048611 A1
(43) Pub. Date: Feb. 13, 2020

(54) ENGINEERED PLATFORM FOR CONNECTED MICROPATTERNED CARDIAC TISSUES

(71) Applicant: Wisconsin Alumni Research Foundation, Madison, WI (US)

(72) Inventors: Wendy Catherine Crone, McFarland, WI (US); Brett Nicholas Napiwocki, Stevens Point, WI (US); Max R. Salick, Madison, WI (US)

(21) Appl. No.: 16/101,125

(22) Filed: Aug. 10, 2018

(51) Int. Cl. C12N 5/077

(52) U.S. Cl. CPC C12N 5/0657; C12N 2535/00; C12N 2533/50

(57) ABSTRACT

Disclosed are compositions and methods for engineering muscle tissue such as cardiac muscle and skeletal muscle. Tissue engineering platforms use a substrate and a protein micropattern. The protein micropattern forms a plurality of protein lanes and at least one protein bridge that connects adjacent protein lanes having a connection angle is less than 90 degrees. Tissue engineering platforms can promote muscle cell alignment and be used for cell-based pharmacological studies. Also disclosed are degradable substrates for transferring a protein micropattern in the form of a plurality of protein lanes and at least one protein bridge that connects adjacent protein lanes having a connection angle is less than 90 degrees.

### BACKGROUND

The present disclosure relates generally to tissue engineering. In particular, the present disclosure relates to compositions and methods for engineering muscle tissue such as cardiac muscle and skeletal muscle. Tissue engineering platforms can promote muscle cell alignment and be used for cell-based pharmacological studies.

Cardiovascular disease is the leading cause of mortality in the United States and many other industrialized nations. Cardiovascular disease leads to over 800,000 deaths and approximately 610,000 new myocardial infarctions occur in the United States each year. Additionally, numerous skeletal muscle diseases impair function and can lead to death. Thus improved models, therapies and treatments for the diseases are needed.

Muscle cells derived from human pluripotent stem cells (hPSC) possess immense therapeutic potential and can serve as innovative pre-clinical platforms for drug development and disease modeling studies; however, methods are lacking to mature hPSC to better reflect the properties of adult cardiomyocytes (CMs) and skeletal muscle cells (SMs) found in vivo.

Induction of cardiac differentiation has been successfully achieved from pluripotent human embryonic stem cell populations. Researchers have produced cardiac phenotypes by applying growth factors Activin A and BMP4 to embryoid bodies and cell monolayers. Improved efficiencies of differentiation have been achieved using small molecule Wnt inhibitors and extracellular matrix sandwich methods. One limitation of these methods is that cardiomyocytes with predominantly immature properties are produced. In particular, rather than becoming organized, aligned “barrel”-shaped cells that are typically seen in native adult myocardium, the human embryonic stem cell (hESC)-derived CMs form into randomly-aligned monolayers or clusters of contracting cells. Additionally, the multinucleation, myosin heavy chain isoform expression, Ca²⁺-handling, and biochemical response characteristics that are ordinarily exhibited by mature adult cardiomyocytes are not achieved by these immature CMs. Recent studies have demonstrated that prolonged culture (60-120 days) of immature cardiomyocytes appears to lead to more mature-like phenotypes. Cardiomyocytes from prolonged cultures displayed increased multinucleation, sarcomere length, and cell size, with reduced Ca²⁺ time-to-peak and decay time. The cardiomyocytes still did not achieve the level of sarcomere structure and alignment observed in native adult cardiomyocytes, however.

Induction of skeletal myogenic progenitors has been successfully achieved from pluripotent human embryonic stem cell populations. Researchers have produced myogenic progenitors using either exogenous expression of myogenic genes such as PAX3, PAX7, and MYOD; small molecules and growth factors; or free-floating spherical culture. However, skeletal muscle derived from stem cells do not achieve the level of sarcomere structure and alignment observed in native adult muscle tissue in vitro.

Human induced pluripotent stem cells (iPSCs) can be generated from both healthy donors and patients with genetic disorders associated with cardiac and skeletal muscle diseases. These cells can be differentiated to the desired myogenic lineage for the purposes of tissue engineering and biological study.

Two-dimensional micropatterning studies and three-dimensional engineered topographies have demonstrated positive results that more accurately mimic the cells' physiological environment while improving throughput, accuracy, and efficiency. Two-dimensional micropatterning studies indicate that the architecture of the extracellular environment influences cell behavior with respect to morphology, migration, cytoskeletal structure, nuclear shape, lineage determination, and functionality. Studies in which neonatal rat and mouse cardiomyocytes were seeded onto two-dimensional micropatterned stripes formed myofibers with improved cell shape, sarcomere organization, cell-cell junction protein expression, contractile strength, and α-catenin expression.

A key function of muscle cells is their ability to contract. To do so efficiently, the cell's internal contractile apparatus must be well organized and aligned. A major limitation of traditional 2-dimensional (2D) myocyte cultures has been the disorganization of the cells' contractile apparatus, which is in marked contrast to the highly aligned and organized features of in vivo muscle tissue.

Methods that promote myocyte alignment have the potential to lead to a new range of studies on human embryonic stem cell-derived cardiomyocytes. Emerging methods of differentiating hPSCs into myocytes show great promise in their repeatability, efficiency, and reduced cost. Previously published protocols continue to only achieve an immature phenotype of myocytes, with limited cell size and elongation, low levels of multinucleation, and immature action potential characteristics. Accordingly, there exists a need for alternative approaches and methods for culturing muscle cells to achieve a more mature phenotype.

### BRIEF DESCRIPTION

The present disclosure is generally related to tissue engineering. In particular, the present disclosure relates to compositions and methods for engineering muscle tissue such as cardiac muscle and skeletal muscle.

In one aspect, the present disclosure is directed to a tissue engineering platform comprising: a substrate; and a protein micropattern, wherein the protein micropattern forms a plurality of protein lanes and at least one protein bridge, wherein the at least one protein bridge connects adjacent protein lanes, and wherein the protein bridge to protein lane connection angle is less than 90 degrees.

In one aspect, the present disclosure is directed to a cell culture method, the method comprising: providing a tissue engineering platform comprising: a substrate; and a protein micropattern, wherein the protein micropattern forms a plurality of protein lanes and at least one protein bridge, wherein the at least one protein bridge connects adjacent protein lanes, and wherein the protein bridge to protein lane connection angle is less than 90 degrees; seeding at least one protein lane with a cell; and culturing the cell.

In one aspect, the present disclosure is directed to a method for preparing a tissue engineering platform, the method comprising: applying a protein onto a substrate surface, wherein the protein forms a plurality of protein lanes and at least one protein bridge, wherein the at least one protein bridge connects adjacent protein lanes, and wherein the protein bridge to protein lane connection angle is less than 90 degrees.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The disclosure will be better understood, and features, aspects and advantages other than those set forth above will become apparent when consideration is given to the following detailed description thereof. Such detailed description makes reference to the following drawings, wherein:

FIG. 1 is a fluorescent micrograph depicting hPSC-CMs grown on micropatterned lanes of varying widths and cultured on 10 kPa PDMS. Actin in red, DAPI in blue. Scale bar=25 μm.

FIGS. 2A and 2B are bright field micrographs depicting hPSC-CMs cultured on 10 kPa PDMS micropatterned meshes. The 30 μm lanes are connected by (A) bridges that are orientated 90 degrees with respect to the lane (FIG. 2A) and 30 μm lanes are connected by bridges that are orientated 15 degrees with respect to the lane (FIG. 2B). Stars indicate areas where CMs detached in the lane and arrows denotes bridge detachment. Scale bar=100 μm.

FIGS. 3A-3D are fluorescent micrograph depicting hPSC-CMs grown on 10 kPa PDMS substrates having micropatterned lanes and bridges (FIGS. 3A and 3C) or unpatterned (FIGS. 3B and 3D). hPSC-CMs cultured on patterned substrates maintain myofibril alignment in the direction of the lane and bridges between lanes. hPSC-CMs cultured on unpatterned substrates have no clear organization and display myofibril disarray. FIGS. 3A and 3B are low magnification images. FIGS. 3C and 3D are high magnification images. Alpha-actinin=green, DAPI=blue.

FIG. 4 is a graph depicting quantification of the percent of myofibrils aligned +/−10% of the superior angle for hPSC-CMs cultured on patterned and unpatterned monolayers. *Statistically significant data using one-way ANOVA with post hoc Tukey tests.

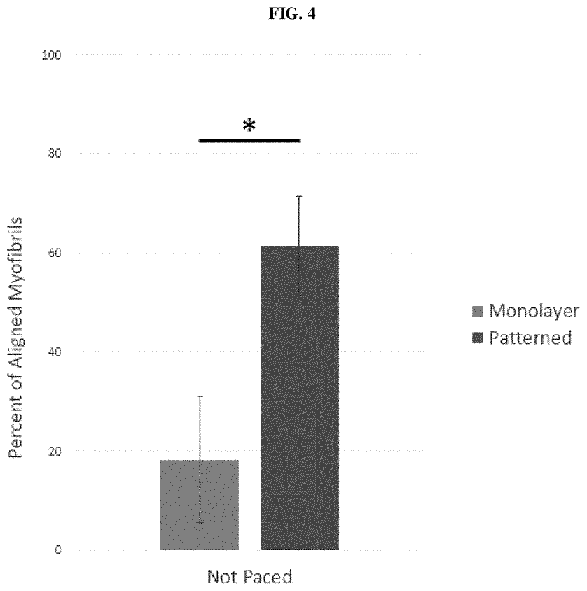

FIGS. 5A-5C depict amplitude of contraction observed for patterned CMs in the presence of 1 μM isoproterenol. FIG. 5A depicts a bright field image of patterned CMs in the same orientation as the images analyzed in FIGS. 5B and 5C. FIG. 5B depicts the x-displacement of CMs under normal contraction and FIG. 5C depicts CMs contracting in the presence of 1 μM isoproterenol. Scale bar goes from +10 μm in red to −10 μm in blue.

FIGS. 6A-6C are illustrations depicting micropatterns of protein lanes connected by 15° protein bridges in the form of a chevron micropattern (FIG. 6A), a mesh micropattern (FIG. 6B), and a diamond micropattern (FIG. 6C). White areas denote where proteins would be present, black areas are void of proteins.

### DETAILED DESCRIPTION

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the preferred methods and materials are described below.

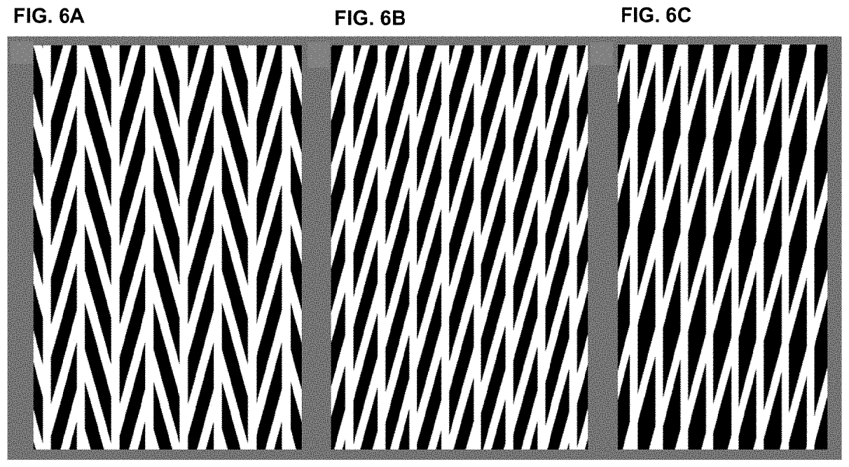

As used herein, the term “micropattern” refers to application of a protein to a substrate to form a shape or design. As used herein, micropattern is not intended to refer only to a repeating pattern. Application of the protein to form lanes of protein and bridges of protein that connect adjacent protein lanes makes an arrangement of the protein into a shape or design. The shape or design of the micropattern can be, for example, in the form diamonds, chevrons, and mesh (see e.g., FIGS. 6A-6C). The shape or design of the micropattern can also be in the form of an

“S-shape” and other curved shapes. Protein lanes with or without protein bridges form a 2-dimensional micropatterned feature for cell adhesion, cell alignment, sarcomere alignment, and maturation.

As used herein, the terms “lane” and “protein lane” refer to an elongated micropattern of protein applied to a substrate having a length and width. Protein lanes can be of any desired width. Particularly suitable protein lane widths are less than 300 μm. Suitably, protein lane widths are less than 250, including less than 150 μm, including less than 100 μm, and including less than 75 μm, including less than 50 μm, less than 40 μm, including less than 30 μm, including less than 20 μm, and including less than 10 μm, and including less than 5 μm. Protein lane widths can range from about 5 μm to about 300 μm, including from about 10 μm to about 250 μm, including from about 150 μm to about 150 μm, including from about 30 μm to about 50 μm. Protein lanes can have varying lengths. Protein lane lengths are generally determined by the substrate size. Suitable protein lane lengths can range from about a few micrometers to more than a few centimeters. Protein lane length can be adjusted to allow for calcium transient analysis and efficient use of the substrate surface. Spacing between protein lanes can also be varied. For example, spacing between protein lanes can generally range from about 10 μm to about 100 μm.

As used herein, the terms “bridge” and “protein bridge” refer to a protein applied to a substrate between adjacent protein lanes that connects adjacent protein lanes. Suitably, the bridge-to-lane connection has an angle less than 90°, including less than 85°, including less than 80°, including less than 75°, including less than 70°, including less than 65°, including less than 60°, including less than 55°, including less than 50°, including less than 45°, including less than 40°, including less than 35°, including less than 30°, including less than 25°, including less than 20°, including less than 15°, including less than 10°, and including less than 5°. Particularly suitable bridge-to-lane connection angles can range from about 5° to about 90°, including about 5° to about 85°, including about 10° to about 85°, including about 10° to about 60°, and including about 15° to about 45°.

Protein lanes and protein bridges can be created on a substrate by methods known in the art. Protein lanes and protein bridges can be directly and indirectly created on a substrate. Suitable methods include, for example, stamping, microcontact printing, manual application (e.g., depositing by pipetting; depositing by syringe; depositing with a paint brush), microfluidics, and bioprinting. Additionally, protein lanes and protein bridges can be indirectly created on a substrate by applying the protein onto a sacrificial layer such as a degradable substrate as described herein that is contacted with another substrate and then the degradable substrate degrades or dissolves. When the sacrificial layer degrades or dissolves, the micropattern of protein from the sacrificial layer is transferred to the substrate.

Suitable proteins for forming lanes and bridges can be extracellular matrix proteins, cell adhesion proteins, and combinations thereof. Protein lanes and protein bridges can be made of the same protein, different proteins, and combinations of proteins. Suitable extracellular matrix proteins include fibronectin, collagen, nidogen, elastin, laminin, heparin sulfate proteoglycans, and combinations thereof. Additionally, MATRIGEL® can be used as the extracellular matrix protein either alone or in combination with fibronectin, collagen, elastin, laminin, and cell adhesion proteins. Suitable cell adhesion proteins include immunoglobulin cell adhesion molecules, cadherins, and integrins. Suitable cell adhesion peptides also and include cell adhesion peptides such as, for example, arginylglycylaspartic acid (RGD). As used herein, a “cell adhesion peptide” refers to an amino acid sequence obtained from a cell adhesion protein to which cells bind via a receptor-ligand interaction.

In one aspect, the present disclosure is directed to a tissue engineering platform comprising: a substrate; and a protein micropattern, wherein the protein micropattern forms a plurality of protein lanes and at least one protein bridge, wherein the at least one protein bridge connects adjacent protein lanes, and wherein the protein bridge to protein lane connection angle is less than 90 degrees.

Suitable substrates include glasses, plastics, polymers, and combinations thereof, and can range from the kPa to the MPa in Young's modulus. Particularly suitable substrates are biocompatible flexible polymers. The polymer stiffness can range from about 5 kPa to about 1.7 MPa. Particularly suitable polymer substrate stiffness ranges from about 5 kPa to about 50 kPa. The polymer can be porous or non-porous. Suitable polymers include synthetic rubbers such as silicone rubbers (e.g., polydimethylsiloxane (PDMS)), polyurethane rubber, styrene butadiene rubber, and acrylonitrile butadiene rubber, natural rubbers (e.g., poly-cis-isoprene), thermoplastic elastomers (e.g., thermoplastic polyurethane, thermoplastic copolyester, thermoplastic polyamide), epoxies (e.g., SU-8), polyimides, polyurethanes, polyamides, polyesters (e.g., poly(lactic-co-glycolic acid) (PLGA), polylactic acid (PLA), polyglycolic acid (PGA), polycaprolactone (PCL), poly(glycerol sebacate) (PGS)), polysaccharides (e.g., chitosan), parylene, and combinations thereof. Advantageously, these polymers are flexible upon application of an applied force. In one particular aspect, the polymer is 10 kPa PDMS.

Suitable proteins are described herein.

Suitable dimensions of the protein lanes and protein bridges are described herein.

Additional features such as intentional gaps in the protein micropatterns can be designed to produce conduction pathways. Gaps are particularly suitable for the study of cardiac arrhythmias, for example.

The tissue engineering platform can further include cells. Suitable cells include muscle cells. Suitable muscle cells include myocytes and myoblasts. Myocytes and myoblasts can be obtained from cardiac muscle and skeletal muscle. Suitable cells also include pluripotent stem cells such as induced pluripotent stem cells (iPSCs), progenitor cells, and combinations thereof. Suitable cells also include fibroblasts, neurons, endothelial cells, and other cell types adjacent to muscle cells. Other cell types that have been shown to produce similar organization and synchronized behavior can also be used. Different cell types can be cultured alone or with other cell types. One cell type can be incorporated into the tissue engineering platform, followed by incorporation of another cell type into the tissue engineering platform after the first cell type. In one embodiment, different cell types can be mixed and incorporated into the tissue engineering platform together (at the same time).

Polymer substrates that are hydrophobic can be treated and/or coated such to render the polymeric substrate hydrophilic. Any methods known in the art for rendering flexible polymers hydrophilic can be used without departing from the scope of the present disclosure. For example, an oxygen plasma treatment on the surface of a polymer can transform the hydrophobic surface to a hydrophilic surface by introducing polar functional groups, which yields a completely wettable surface. Accordingly, methods for surface treatment include oxygen plasma treatment, ultraviolet (UV) radiation, UV/ozone treatment, corona discharges, as well as certain types of polymer or co-polymer coatings as known in the art.

In another aspect, the present disclosure is directed to a cell culture method. The method includes: providing a tissue engineering platform comprising: a substrate; and a protein micropattern, wherein the protein micropattern forms a plurality of protein lanes and at least one protein bridge, wherein the at least one protein bridge connects adjacent protein lanes, and wherein the protein bridge to protein lane connection angle is less than 90 degrees; seeding at least one lane with a cell; and culturing the cell.

Suitable cells are described herein. Different cell types can be cultured alone or with other cell types. One cell type can be incorporated into the tissue engineering platform, followed by incorporation of another cell type into the tissue engineering platform after the first cell type. In one embodiment, different cell types can be mixed and incorporated into the tissue engineering platform together (at the same time). In one embodiment, a muscle cell, a pluripotent stem cell, a progenitor cell, and combinations thereof, is co-cultured with a fibroblast cell, an endothelial cell, a neural cell, and combinations thereof. For example, a muscle cell can be co-cultured with a fibroblast cell, an endothelial cell, a neural cell, and combinations thereof. Similarly, a pluripotent stem cell can be co-cultured with a fibroblast cell, an endothelial cell, a neural cell, and combinations thereof. A progenitor cell can be co-cultured with a fibroblast cell, an endothelial cell, a neural cell, and combinations thereof.

The method can further include applying suitable cues to influence cell behavior and enhance maturation. Suitable cues include, for example, biochemical factors, mechanical stimulation and electrical stimulation. Biochemical factors include agents such as growth factors that can cause cells to differentiate. For example, biochemical factors can be used in the method to cause pluripotent stem cells and a progenitor cells to differentiate into muscle cells. For electrical simulation, cyclic stimulation can be applied at frequencies ranging from about 0.25 Hz to about 2 Hz.

Suitable extracellular matrix proteins and cell adhesion proteins are described herein.

Suitable substrates are described herein.

The method can further include contacting the cell with a candidate agent to study its influence on cell behavior. Suitable candidate agents include drugs, biologics, toxins, and combinations thereof.

The method can further include the use of measurement techniques that can be directly applied. The method is amenable to standard microscopy techniques that produce live imaging for direct analysis with techniques such as digital image correlation, immunohistochemistry imaging of the cells and proteins, and incorporation of fluorescent beads in the polymer substrate to enable traction force microscopy.

In one aspect, the present disclosure is directed to a method for preparing a tissue engineering platform. The method includes applying a protein onto a substrate surface, wherein the protein forms a plurality of protein lanes and at least one protein bridge, wherein the at least one protein bridge connects adjacent protein lanes, and wherein the protein bridge to protein lane connection angle is less than 90 degrees.

Suitable methods for applying the protein in the micropattern to form protein lanes and protein bridges are described herein.

Suitable extracellular matrix proteins and adhesive peptides are described herein.

Suitable substrates are described herein.

Suitable protein lane and protein bridge features are described herein.

The method can further include applying suitable cues to influence cell behavior and enhance maturation. Suitable cues include, for example, biochemical factors, mechanical stimulation and electrical stimulation. Devices for providing mechanical and electrical stimulation to the tissue engineering platform can be connected to the tissue engineering platform.

Advantageously, the tissue engineering platforms of the present disclosure can be used for cell culturing, transplantation, developmental modeling, disease modeling, and for drug screening. Use of the tissue engineering platform results in the formation of an organized multi-cellular construct that mimics the cellular structure and organization observed in vivo.

When the tissue engineering platform of the present disclosure is used for drug screening or toxicity assays, candidate agents can be added to a culture media. Cell health and survival can then be assessed using standard techniques.

In one aspect, the present disclosure is directed to a degradable substrate for transferring a protein micropattern comprising: a degradable substrate and a protein micropattern, wherein the protein micropattern forms a plurality of protein lanes and at least one protein bridge, wherein the at least one protein bridge connects adjacent protein lanes, and wherein the protein bridge to protein lane connection angle is less than 90 degrees.

The degradable substrate for transferring a protein micropattern can be used to transfer the protein micropattern to another suitable substrate. Other suitable substrates can be, for example, glasses, plastics, and polymers. For example, the degradable substrate for transferring a protein micropattern can be positioned onto a glass microscope coverslip. Upon degradation of the degradable substrate, the protein is transferred onto the surface of the glass microscope coverslip in the micropattern of the degradable substrate. Similarly, the degradable substrate for transferring a protein micropattern can be positioned onto a plastic cell culture dish such as a 96-well plate. Upon degradation of the degradable substrate, the protein is transferred onto the surface of the plastic dish in the micropattern of the degradable substrate.

Suitable degradable substrate materials are biocompatible degradable substrates. Particularly suitable degradable substrates are degradable films. Suitable substrate materials include any material that can be made into a rigid or semi-rigid film to which a protein micropattern can be applied (such as by stamping, microcontact printing, manual application (e.g., depositing by pipetting; depositing by syringe; depositing with a paint brush), microfluidics, and bioprinting, as described herein and known to those skilled in the art). The degradable substrate material should also maintain fidelity of the micropattern as it is applied to another substrate for transferring the protein micropattern. The substrate material should also have the capability of being dissolved or degraded without denaturing the protein of the protein micropattern and also not leave a residue that interferes with cell survival, cell attachment, cell spreading, cell migration, cell differentiation, and cell growth. Suitable substrate materials can be, for example, a polyurethane, a poly(ester amide), polylactic acid, poly(caprolactone), polyvinyl alcohol, polyvinylpyrrolidone, poly(lactic-co-glycolic) acid, polyglycolic acid, polyglycolide, polylactide, polyhydroxobutyrate, chitosan, hyaluronic acid, poly(2-hydroxyethyl-methacrylate), poly(ethylene glycol), sugars or starches (e.g., polymeric carbohydrates) and combinations thereof.

The degradable substrate for transferring a protein micropattern can be prepared by applying a protein onto the substrate to form a protein micropattern in which the protein micropattern is in the form of protein lanes and protein bridges. Protein bridges form connections between protein lanes. The protein can be applied by any suitable method such as for example, stamping, microcontact printing, manual application (e.g., depositing by pipetting; depositing by syringe; depositing with a paint brush), microfluidics, and bioprinting.

### EXAMPLES

### Materials and Methods

### Example 1

In this Example, cardiomyocytes were differentiated from the 19-9-11 iPSC line and purified using lactate media. 10 kPa PDMS substrates mimicking the elasticity of the healthy myocardium were made by blending together commercially available Sylgard 184 and 527. Microcontact printing on soft substrates was achieved using a sacrificial polyvinyl alcohol (PVA) film that resulted in the precise pattern transfer of proteins from the film to the soft substrate. Statistical analyses were done using one-way ANOVA with post hoc Tukey test.

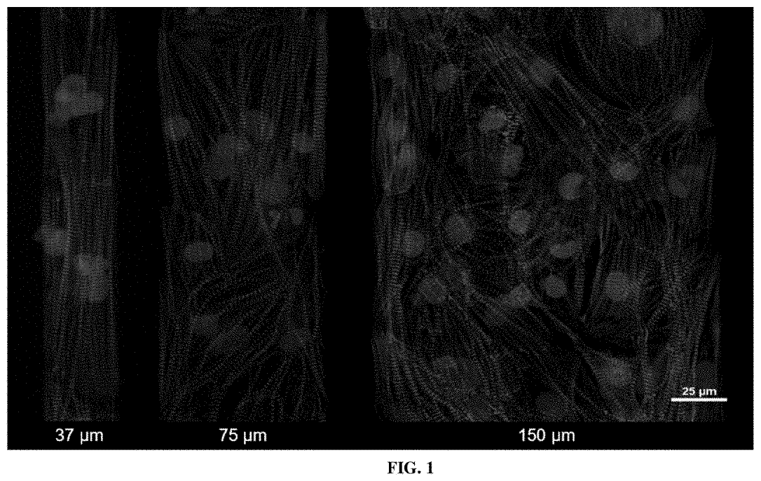

The cytoskeletal structure of hPSC-CMs have an adult-like phenotype when micropatterned onto soft substrates (FIG. 1). Lane widths under 40 μm resulted in myofibrils that aligned with the direction of the lane.

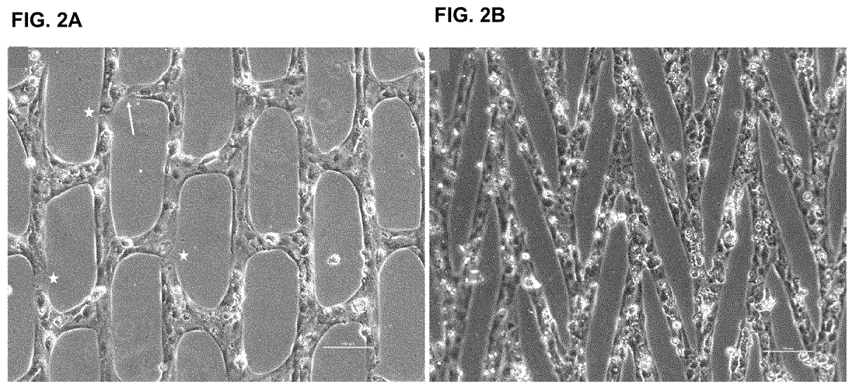

While smaller lane widths controlled myofibril alignment, each CM lane contracted independently of adjacent micropatterned lanes. To induce synchronized contraction over large 2D regions while maintaining the organization produced by the lane edges, micropatterned ECM bridges were added between lanes to form a connected tissue. 90 degrees and 15 degrees angles of bridge-to-lane connection were analyzed (FIGS. 2A and 2B, respectively). 90 degree bridges that were perpendicular to the lanes resulted in degradation of cells near the bridges during CM culture. Over time the connection between CMs would break as bridge CMs generated stress in the direction perpendicular to those in the lanes causing the CMs to lose connections and pull away from one another. The 15 degree patterns remained intact for duration of the culture period (18 days) resulting in synchronous contraction over the entire ECM micropattern.

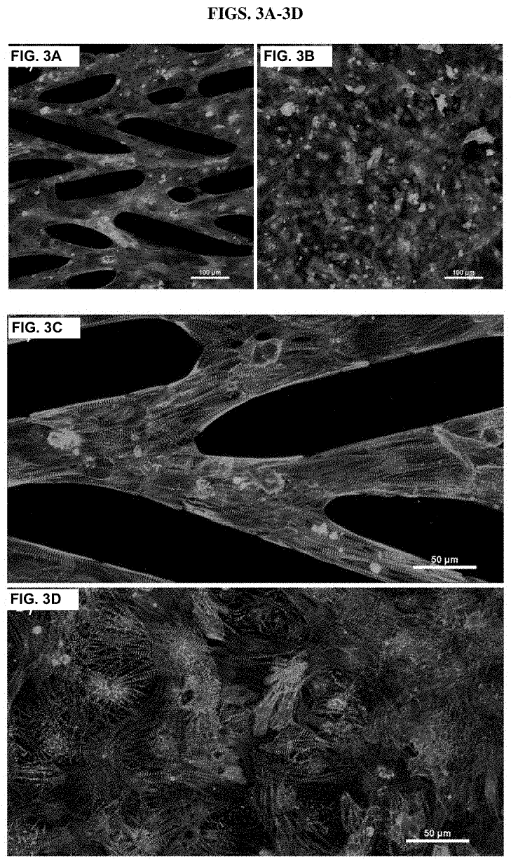

As shown in FIG. 3, the patterned samples produced significantly more aligned myofibrils than the unpatterned controls (compare FIGS. 3A and 3C with FIGS. 3B and 3D). Unlike monolayers which displayed isotropic electrical impulse propagation, hPSC-CMs grown on micropatterned substrates demonstrated anisotropic electrical impulse propagation, as occurs in the native myocardium.

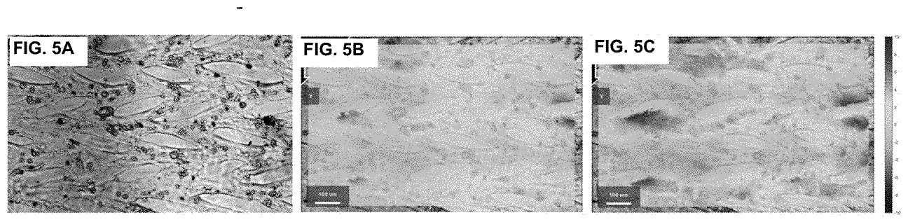

The utility of the engineered anisotropic cardiac tissues to serve as innovative platforms for drug discovery was demonstrated by beta-adrenergic stimulation with isoproterenol. An increase in the rate of contraction was observed for patterned CMs in the presence of 1 μM isoproterenol. Additionally, the amplitude of contraction also increased from baseline as indicated by larger displacement values when going from a normal contraction to a contraction in the presence of isoproterenol (FIGS. 5B and 5C, respectively).

The results presented herein used microcontact printing, soft substrates and electrical stimulation to improve CM maturation. The 15 degree micropattern angle allowed the patterned CMs to stay connected for the duration of the experiment. Functional studies including digital image correlation measuring displacements and strains indicated improvements in maturation and the correct physiological response to beta-adrenergic stimulation. These results highlight the benefit of using multiple signaling cues when engineering cardiac tissues to better mimic the native human heart and create more representative models for cardiac assays.

When introducing elements of aspects of the disclosure or the examples thereof, the articles “a,” “an,” “the,” and “said” are intended to mean that there are one or more of the elements. Furthermore, references to an “embodiment” or “example” of the present disclosure are not intended to be interpreted as excluding the existence of additional embodiments or examples that also incorporate the recited features. The terms “comprising,” “including,” and “having” are intended to be inclusive and mean that there may be additional elements other than the listed elements. The phrase “one or more of the following: A, B, and C” means “at least one of A and/or at least one of B and/or at least one of C.”

Having described aspects of the disclosure in detail, it will be apparent that modifications and variations are possible without departing from the scope of aspects of the disclosure as defined in the appended claims. As various changes could be made in the above constructions, products, and methods without departing from the scope of aspects of the disclosure, it is intended that all matter contained in the above description and shown in the accompanying drawings shall be interpreted as illustrative and not in a limiting sense.

While the aspects of the disclosure have been described in terms of various examples with their associated operations, a person skilled in the art would appreciate that a combination of operations from any number of different examples is also within the scope of the aspects of the disclosure.

What is claimed is:

1. A tissue engineering platform comprising:
a substrate; and
a protein micropattern, wherein the protein micropattern forms a plurality of protein lanes and at least one protein bridge, wherein the at least one protein bridge connects adjacent protein lanes, and wherein the protein bridge to protein lane connection angle is less than 90 degrees.

2. The tissue engineering platform of claim 1, wherein the substrate is selected from the group consisting of a glass, a plastic, a polymer, and combinations thereof.

3. The tissue engineering platform of claim 1, wherein the protein lane width is less than 300 μm.

4. The tissue engineering platform of claim 1, wherein the protein is selected from the group consisting of an extracellular matrix protein, a cell adhesion protein, and combinations thereof.

5. The tissue engineering platform of claim 1, wherein the protein micropattern is in a shape selected from the group consisting of a diamond micropattern, a chevron micropattern, a mesh micropattern, and a curved micropattern.

6. The tissue engineering platform of claim 1, further comprising a cell.

7. The tissue engineering platform of claim 1, wherein the protein bridge to protein lane connection angle is about 15 degrees.

8. The tissue engineering platform of claim 1, wherein the protein lane width is less than 40 μm.

9. A cell culture method, the method comprising:
providing a tissue engineering platform comprising: a substrate; and a protein micropattern, wherein the protein micropattern forms a plurality of protein lanes and at least one protein bridge, wherein the at least one protein bridge connects adjacent protein lanes, and wherein the protein bridge to protein lane connection angle is less than 90 degrees;
seeding at least one protein lane with a cell; and
culturing the cell.

10. The method of claim 9, wherein the cell is selected from the group consisting of a muscle cell, a pluripotent stem cell, a progenitor cell, and combinations thereof.

11. The method of claim 10, further comprising a fibroblast cell, an endothelial cell, a neural cell, and combinations thereof.

12. The method of claim 9, wherein the protein is selected from the group consisting of an extracellular matrix protein, a cell adhesion protein, and combinations thereof.

13. The method of claim 9, further comprising providing a stimulation selected from the group consisting of biochemical stimulation, mechanical stimulation, electrical stimulation, and combinations thereof.

14. The method of claim 9, further comprising contacting the cell with a candidate agent.

15. The method of claim 9, wherein the protein bridge to protein lane connection angle is about 15 degrees.

16. The method of claim 9, wherein the protein lane width is less than 40

17. A method for preparing a tissue engineering platform, the method comprising:
applying a protein onto a substrate surface, wherein the protein forms a plurality of protein lanes and at least one protein bridge, wherein the at least one protein bridge connects adjacent protein lanes, and wherein the protein bridge to protein lane connection angle is less than 90 degrees.

18. The method of claim 17, wherein the protein is selected from the group consisting of an extracellular matrix protein, a cell adhesion protein, and combinations thereof.

19. The method of claim 17, wherein the protein lane width is less than 300 μm.

20. The tissue engineering platform of claim 17, wherein the protein bridge to protein lane connection angle is about 15 degrees.
